(19) United States
(12) Patent Application Publication
(10) Pub. No.: US 2020/0197187 A1
(43) Pub. Date: Jun. 25, 2020

(54) ARTIFICIAL ANKLE JOINT TALUS COMPONENT

(71) Applicants: CORENTEC CO., LTD., Cheonan-si, Chungcheongnam-do (KR); CHONNAM NATIONAL UNIVERSITY HOSPITAL, Gwangju (KR); INDUSTRY FOUNDATION OF CHONNAM NATIONAL UNIVERSITY, Gwangju (KR)

(72) Inventors: Keun-Bae Lee, Gwangju (KR); Tae-Jin Shin, Hanam-si, Gyeonggi-do (KR)

(21) Appl. No.: 16/642,745

(22) PCT Filed: Aug. 28, 2018
(86) PCT No.: PCT/KR2018/009913

§ 371 Date: Feb. 27, 2020

(30) Foreign Application Priority Data
Aug. 29, 2017 (KR) 10-2017-0109175

(51) Int. Cl. A61F 2/42

(52) U.S. Cl. CPC A61F 2/4202; A61F 2002/3093; A61F 2002/4207

(57) ABSTRACT

The present disclosure relates to an artificial ankle joint talus component and, more particularly, to an artificial ankle joint talus component including a joint surface in contact with an insert and a contact surface in contact with a bone, wherein the contact surface may be formed to be complementary to a resected surface of a talus so as to cover the entire resected surface, thereby dispersing stress, and reducing after-effects of surgery, such as osteolysis, heterotopic ossification, or the like.

### BACKGROUND OF THE INVENTION

### 1. Field of the invention

The present disclosure relates to an artificial ankle joint talus component and, more particularly, to an artificial ankle joint talus component including a joint surface in contact with an insert and a contact surface in contact with a bone, wherein the contact surface is formed to be complementary to a resected surface of a talus so as to cover the entire resected surface, thereby dispersing stress, and reducing the after-effects of surgery, such as osteolysis, heterotopic ossification, or the like.

### 2. Description of the Prior Art

In the case where the ankle joint fails to execute its intrinsic function due to various causes such as degenerative arthritis, post-traumatic arthritis, rheumatoid arthritis, and the like of an ankle, arthroplasty is performed using an artificial ankle joint. Clinic trials for artificial ankle joint arthroplasty, which started in the 1970s did not meet expectations because many complications occurred in early stages, and were found to increase the burden on the surgeon because the surgery procedure was highly complicated. Therefore, artificial ankle joint arthroplasty tends to be avoided, and treatment was most often performed using an ankle fusion procedure. However, the development of replacements and the development of surgical methods have brought about satisfactory clinical results, and patient's satisfaction therewith has risen, and thus joint arthroplasty is widespread today.

There are various kinds of artificial ankle joints, and, a three-component mobile-bearing type, which primarily includes a tibial prosthesis coupled to a tibia, a talus prosthesis coupled to a talus, and an insert serving as a bearing by connecting the above two components, is the most widely used joint in Korea.

In general, in order to implant a talus prosthesis into a talus 91, the fornix of the talus is resected within a certain range so as to correspond to the contact surface of a talus prosthesis, and a talus prosthesis is coupled to the resected surface. Here, it is preferable to minimize the amount of bone to be removed in consideration of revision arthroplasty.

### PRIOR PATENT

EP 2124822 (Aug. 8, 2001. Registration) “IMPROVEMENT AND RELATING TO AN ANKLE PROSTHESIS”

The disclosure illustrated in the above prior patent shows that a contact surface includes three planes in order to minimize the amount of bone to be removed. However, since the boundary of the anterior and the posterior is recessed inwards and is thus concave, the resected surface of the talus cannot be fully covered. Therefore, a cross-section of the talus may be exposed in the boundary of the contact surface, and the exposed resected surface may be consistently stimulated by joint liquid, thereby causing osteolysis or heterotopic ossification, in which unnecessary bone is produced.

In general, it is known that osteolysis are caused by joint liquid that infiltrates into the bone through a portion that is not covered by a prosthesis in the resected surface of the bone. In most cases, osteolysis does not show symptoms in the early stage, but is accompanied by pain as it progresses. In addition, osteolysis causes dissociation between a bone and a prosthesis, thereby adversely affecting the lifespan of the artificial joint and, in severe cases, leading to fractures of bone surrounding the artificial joint. Progressive osteolysis requires additional bone grafting, and also requires revision arthroplasty in cases where the reliability of an implant is affected.

Heterotopic ossification indicates that bone tissues are formed in abnormal locations, and often occurs around the joints. If heterotopic ossification occurs after artificial ankle joint arthroplasty, a bone grows around the implant, causing joint pain and restricting joint motion, which may lead to loss of function of the artificial joint.

As can be seen in FIG. 1, heterotopic ossification occurs in the hatched portions “A”, which are not covered by a talus prosthesis, in the centers of the anterior and posterior of the talus prosthesis. This is due to the fact that the anterior and posterior boundaries of the conventional talus prosthesis are formed to be recessed inwards because the central portion thereof is recessed in the form of a groove in order to guide an insert while maintaining a curvature in the anterior and posterior direction in the structure thereof.

The occurrence rate of heterotopic ossification after artificial ankle joint arthroplasty is reported to differ depending on surgeons, but is usually about 25%. In particular, recent research results have reported that heterotopic ossification is accompanied by symptoms in about 5% of patients, thereby limiting the range of motion of a joint and causing severe pain in the joints, so that the function of the artificial joint significantly deteriorates. In order to resolve this inconvenience, a surgical method of removing the generated bones is required, which further imposes a burden of reoperation on the patient.

However, a large talus prosthesis cannot be used indeliberately in order to avoid the above problem. The talus has an anatomical structure in which the anterior portion is wider than the posterior portion. An existing talus prosthesis has the same width in the anterior portion and the posterior portion, so that the shape of the prosthesis does not conform to the talus. Accordingly, the posterior portion of the talus prosthesis may collide with a medial/lateral malleolus. Therefore, in order to avoid interference between the talus prosthesis and the posterior portion of the malleolus, a slightly smaller implant is generally used, whereas a big implant must be used to cover the resected surface of the bone, which makes it difficult to decide the size of the implant. Therefore, the above problem can be solved by changing the shape of the talus prosthesis, instead of selecting the size of the implant. In particular, clinically, osteolysis primarily occurs in the anterior site, and heterotopic ossification frequently occurs in the posterior site. Thus, the front and rear structures of the talus prosthesis must be improved.

As described above, whether or not osteolysis or heterotopic ossification occurs around an implant is an important factor in determining the lifespan and success of the artificial ankle joint. Therefore, an artificial ankle joint having a structure preventing osteolysis or heterotopic ossification is required.

In addition, referring to the disclosure disclosed in the patent document, since the anterior and posterior boundaries are recessed inwards, the insert serving as a bearing is limited in its movable range. In general, a normal ankle joint moves in the range of dorsiflexion of 20 degrees and plantar flexion of 40 to 50 degrees, and an artificial ankle joint must provide a motion in the range of dorsiflexion of at least 10 degrees and plantar flexion of at least 20 degrees after artificial ankle joint arthroplasty. However, since the insert and the talus prosthesis cannot form a joint surface in the final range of the dorsiflexion and plantar flexion in the disclosure of the patent document, the insert is worn out and the flexion range thereof is limited, thereby causing great discomfort to the patient and lowering patient's satisfaction.

Therefore, an artificial ankle joint having a structure capable of securing a full movable range is required.

Lastly, the talus prosthesis of the disclosure disclosed in the patent document includes a peg to be fixed to a talus, and the peg is provided in the anterior portion of the contact surface in which the talus prosthesis comes into contact with the talus. However, in the case where the peg is positioned in the anterior portion, when impacting the talus prosthesis in order to fix the prosthesis to the talus, the posterior portion of the talus prosthesis lifts up and does not contact with the talus, thereby lowering fixing force. Such a structure can be seen in the prior art shown in FIG. 2.

Since the ankle is small, compared to other joints, a surgical site is also very narrow. In addition, an anterior approach method in which the anterior portion of the ankle joint is incised is primarily used in ankle joint arthroplasty. In this case, the incision range is narrower when approaching the surgical site from the anterior side. Thus, it is difficult to determine a correct position for applying an impact force through the narrow incision site in the operation procedure. In particular, in the case where the peg (B) is positioned in the anterior portion, the surgeon must apply force from the side when striking the implant, which causes inconvenience to the surgeon in making a posture and errors according thereto.

If a force acting point is out of a correct position as described above, the posterior portion of the implant lifts up from the talus, which is more severe in the case where the peg (B) is located in the anterior portion (see “C”). In addition, although the implant is made of a rigid metal, it is not a strong body. Thus, even when the force is applied to a correct position, force may be concentrated on the anterior portion, which may bring about slight deformation of the implant so that the posterior portion thereof cannot be securely placed on the talus. As described above, since the peg (B) provided in the anterior portion is distant from the posterior portion, if the implant is not in correct contact with the talus, a firm connection between the talus and the implant cannot be obtained. Even if the posterior portion of the implant is pressed down by the weight of a body after surgery so as to come into contact with the talus, correct placement of the implant cannot be guaranteed.

Actually, such a phenomenon often occurs during surgery, and must always be checked using an image intensifier, thereby increasing the risk of radiation exposure and infection of a surgeon.

Therefore, there is a need for an artificial ankle joint having a structure capable of preventing the phenomenon in which the posterior portion of the talus prosthesis lifts up when impacting the same and guiding the talus prosthesis to be correctly placed on the talus.

### SUMMARY OF THE INVENTION

The present disclosure has been made to solve the above problems, and an objective thereof is to provide an implant that conforms to the anatomical shape of a bone, thereby preventing heterotopic ossification after surgery and distributing stress.

In addition, another objective of the present disclosure is to provide an implant that conforms to the anatomical shape of the talus, thereby preventing heterotopic ossification after surgery and distributing stress.

In addition, another objective of the present disclosure is to provide an implant capable of stably guiding an insert and preventing luxation thereof.

In addition, another objective of the present disclosure is to provide an implant that conforms to the anatomical shape of a posterior portion of a talus, thereby preventing heterotopic ossification after surgery and distributing stress.

In addition, another objective of the present disclosure is to provide an implant that conforms to the anatomical shape of an anterior portion of a talus, thereby preventing osteolysis after surgery and distributing stress.

In addition, another objective of the present disclosure is to provide an implant that conforms to the overall anatomical shape of a talus, thereby preventing osteolysis and heterotopic ossification around the implant after surgery and distributing stress.

In addition, another objective of the present disclosure is to provide an implant capable of minimizing the amount of bone to be removed, thereby facilitating an operation in revision arthroplasty.

In addition, another objective of the present disclosure is to provide an implant capable of reducing surgery time and facilitating surgery by preserving medial and lateral surfaces of the talus, instead of resecting the same.

Further, another objective of the present disclosure is to provide an implant capable of easy insertion in the case of artificial ankle joint arthroplasty using an anterior approach method.

The present disclosure is implemented by embodiments having the following configuration in order to attain the above objectives.

According to an embodiment of the present disclosure, an artificial ankle joint talus component of the present disclosure may include a contact surface having a shape complementary to a resected surface of a bone into which the implant is implanted so as to increase a contact area with the bone, thereby distributing stress and mitigating side effects after surgery.

According to another embodiment of the present disclosure, an artificial ankle joint talus component of the present disclosure may be a talus component coupled to a talus in artificial ankle joint arthroplasty, wherein the talus component may include a joint surface in contact with an insert, and wherein the joint surface is formed to have a curvature in an anterior and posterior direction so as to enable joint motion of an ankle.

According to another embodiment of the present disclosure, an artificial ankle joint talus component of the present disclosure may include only an upper joint surface in contact with an insert without a medial joint surface positioned at a medial side or a lateral joint surface positioned at a lateral side.

According to another embodiment of the present disclosure, there is provided an artificial ankle joint talus component of the present disclosure in which a posterior boundary of the contact surface may have a gentle arc shape, which is formed to be convex toward the posterior, and in which the joint surface may extend to the posterior boundary of the contact surface toward the posterior while having a curvature to maintain a large contact area with the talus, thereby distributing stress, mitigating side effects after surgery, and enabling motion throughout a wide range.

According to another embodiment of the present disclosure, there is provided an artificial ankle joint talus component of the present disclosure in which an anterior boundary of the contact surface may have a gentle arc shape, which is formed to be convex toward the anterior, and in which the joint surface may extend to the anterior boundary of the contact surface toward the anterior while having a curvature to maintain a large contact area with the talus, thereby distributing stress, mitigating side effects after surgery, and enabling motion throughout a wide range.

According to another embodiment of the present disclosure, there is provided an artificial ankle joint talus component of the present disclosure in which a posterior boundary of the contact surface may have a gentle arc shape, which is formed to be convex toward the posterior, in which the joint surface may extend to the posterior boundary of the contact surface toward the posterior while having a curvature, in which an anterior boundary of the contact surface may have a gentle arc shape, which is formed to be convex toward the anterior, and in which the joint surface extends to the anterior boundary of the contact surface toward the anterior while having a curvature to maintain a large contact area with the talus, thereby distributing stress, mitigating side effects after surgery, and enabling motion throughout a wide range.

According to another embodiment of the present disclosure, there is provided an artificial ankle joint talus component of the present disclosure in which tangents of a posterior medial boundary of the medial joint surface and a posterior lateral boundary of the lateral joint surface may continuously extend toward the connection joint surface while having slopes opposite to each other, and may then lead to each other.

According to another embodiment of the present disclosure, there is provided an artificial ankle joint talus component of the present disclosure in which tangents of an anterior medial boundary of the medial joint surface and an anterior lateral boundary of the lateral joint surface may continuously extend toward the connection joint surface while having slopes opposite to each other, and may then lead to each other.

According to another embodiment of the present disclosure, there is provided an artificial ankle joint talus component of the present disclosure in which tangents of a posterior medial boundary of the medial joint surface and a posterior lateral boundary of the lateral joint surface may continuously extend toward the connection joint surface while having slopes opposite to each other, and may then lead to each other, and in which tangents of an anterior medial boundary of the medial joint surface and an anterior lateral boundary of the lateral joint surface may continuously extend toward the connection joint surface while having slopes opposite to each other, and may then lead to each other.

According to another embodiment of the present disclosure, there is provided an artificial ankle joint talus component of the present disclosure in which the slope of the tangent of each boundary may approximate zero as it approaches the connection joint surface.

According to another embodiment of the present disclosure, there is provided an artificial ankle joint talus component of the present disclosure in which the talus component may be formed in a truncated cone shape in which the width thereof increases moving from the posterior to the anterior thereof so as to have a shape complementary to the resected surface of the talus, thereby implementing an anatomical shape, minimizing the amount of bone to be removed, increasing the contact area to distribute stress, and mitigating side effects after surgery.

According to another embodiment of the present disclosure, there is provided an artificial ankle joint talus component of the present disclosure in which the contact surface may include an anterior surface inclined to one side in the anterior portion, an intermediate surface formed in a plane at the center thereof, and a posterior surface inclined to one side in the posterior portion, thereby minimizing the amount of bone to be removed.

According to another embodiment of the present disclosure, an artificial ankle joint talus component of the present disclosure may further include a peg extending from the intermediate surface to a distal end.

According to another embodiment of the present disclosure, there is provided an artificial ankle joint talus component of the present disclosure in which the peg may extend to be inclined at a predetermined angle toward the posterior.

According to another embodiment of the present disclosure, there is provided an artificial ankle joint talus component of the present disclosure in which the peg may be inclined at 60 to 70 degrees toward the posterior.

The present disclosure can give the following effects by the above embodiments, configurations, combinations, and usage relationships, which will be described below.

The present disclosure has the effect of preventing osteolysis and heterotopic ossification after surgery and distributing stress by means of an implant that conforms to the anatomical shape of a bone.

In addition, the present disclosure has the effect of preventing osteolysis and heterotopic ossification after surgery and distributing stress by means of an implant that conforms to the anatomical shape of a talus.

In addition, the present disclosure has the effect of stably guiding an insert and preventing luxation thereof in a joint motion.

In addition, the present disclosure has the effect of preventing osteolysis and heterotopic ossification after surgery and distributing stress by means of an implant that conforms to the anatomical shape of a posterior portion of a talus.

In addition, the present disclosure has the effect of preventing osteolysis and heterotopic ossification after surgery and distributing stress by means of an implant that conforms to the anatomical shape of an anterior portion of a talus.

In addition, the present disclosure has the effect of preventing osteolysis and heterotopic ossification after surgery and distributing stress by means of an implant that conforms to the overall anatomical shape of a talus.

In addition, the present disclosure has the effect of facilitating revision arthroplasty by minimizing the amount of bone to be removed.

In addition, the present disclosure has the effect of preventing the posterior portion of an implant from being lifted up when impacting the same for fixation, thereby allowing the entire implant to come into uniform contact with a resected surface.

In addition, the present disclosure has the effect of facilitating insertion of an implant into the bone when performing artificial ankle joint arthroplasty and preventing the rotation of the implant in the process of inserting the same.

Further, the present disclosure has the effect of facilitating insertion of an implant into the bone when performing artificial ankle joint arthroplasty using an anterior approach method.

### BRIEF DESCRIPTION OF THE DRAWINGS

The above and other aspects, features, and advantages of the present disclosure will be more apparent from the following detailed description taken in conjunction with the accompanying drawings, in which:

FIG. 1 is a plan view showing the state in which a talus component is coupled to a talus according to the prior art;

FIG. 2 is a side view showing the case where a posterior portion of an implant lifts up when impacting a talus component with respect to a talus using a peg according to the prior art;

FIG. 3 is a plan view and a side view showing the position and anatomy of a talus;

FIG. 4 is a side view showing the state in which a talus component is coupled to a tibial component and an insert according to an embodiment of the present disclosure;

FIG. 5 is a perspective view showing a talus component according to an embodiment of the present disclosure;

FIG. 6 is a perspective view showing a talus component according to an embodiment of the present disclosure;

FIG. 7 is a perspective view showing a talus component according to an embodiment of the present disclosure;

FIG. 8 is a plan view showing a talus component according to an embodiment of the present disclosure;

FIG. 9 is a front view showing a talus component according to an embodiment of the present disclosure;

FIG. 10 is a rear view showing a talus component according to an embodiment of the present disclosure;

FIG. 11 is a side view showing a talus component according to an embodiment of the present disclosure;

FIG. 12 is a bottom view showing an intermediate surface according to an embodiment of the present disclosure;

FIG. 13 is a bottom view showing an anterior surface according to an embodiment of the present disclosure;

FIG. 14 is a bottom view showing a posterior surface according to an embodiment of the present disclosure;

FIG. 15 is a plan view showing the state in which a talus component is coupled to a talus according to an embodiment of the present disclosure;

FIG. 16 is a side view showing the movable range of an insert while a talus component is coupled to a talus according to an embodiment of the present disclosure;

FIG. 17 is a cross-sectional side view showing the comparison of a talus component according to the prior art and a talus component according to an embodiment of the present disclosure when a force is incorrectly applied thereto in the process of coupling the talus components to a talus; and

FIG. 18 is a view of radiation photos showing heterotopic ossification that may occur in artificial ankle joint arthroplasty according to the prior art.

### DETAILED DESCRIPTION OF THE EXEMPLARY EMBODIMENTS

Hereinafter, an artificial ankle joint talus component according to the present disclosure will be described in detail with reference to the accompanying drawings. It should be noted that the same components in the figures are represented by the same reference numerals wherever possible. In addition, detailed descriptions of well-known functions and configurations that may unnecessarily obscure the subject matter of the present disclosure will be omitted. Unless otherwise defined, all terms in this specification are equivalent to the general meanings of the terms understood by those of ordinary skill in the art to which the present disclosure pertains, and if the terms conflict with the meanings of the terms used herein, the definition is to be understood according to the present specification.

Now, an artificial ankle joint talus component of the present disclosure will be described in detail with reference to the accompanying drawings.

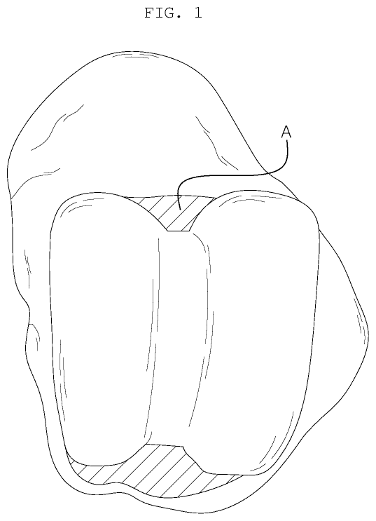

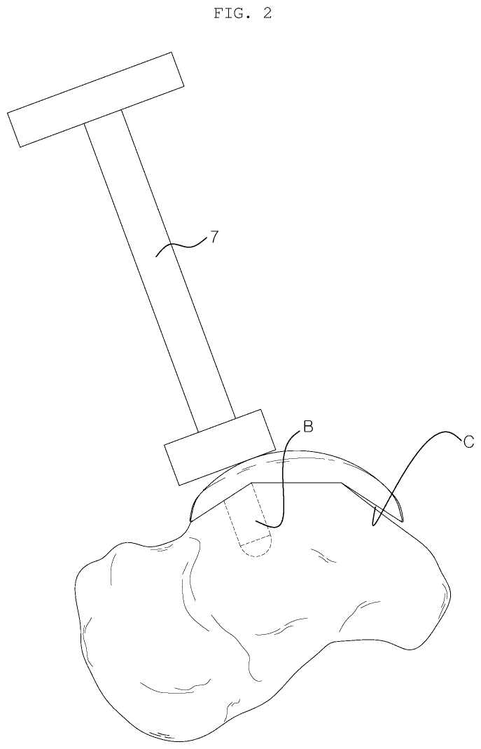

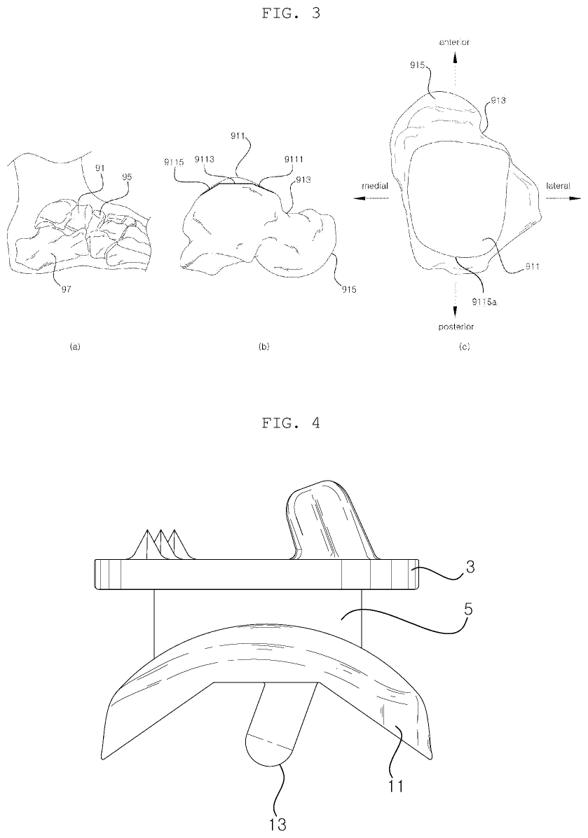

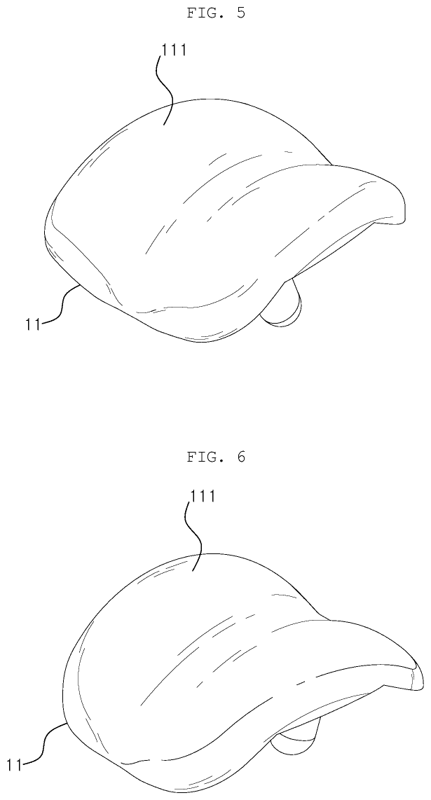

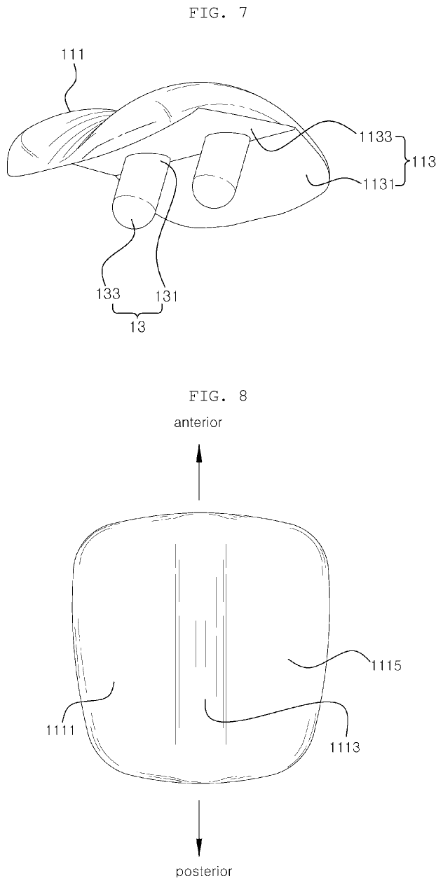

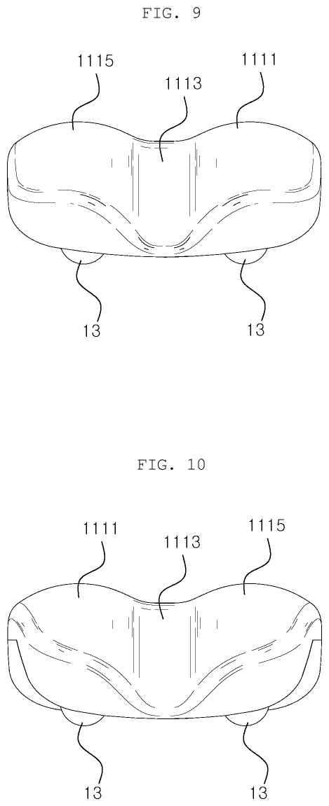

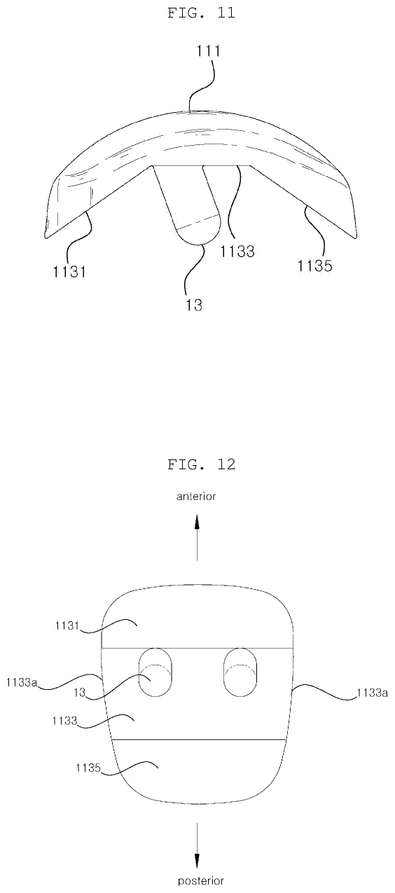

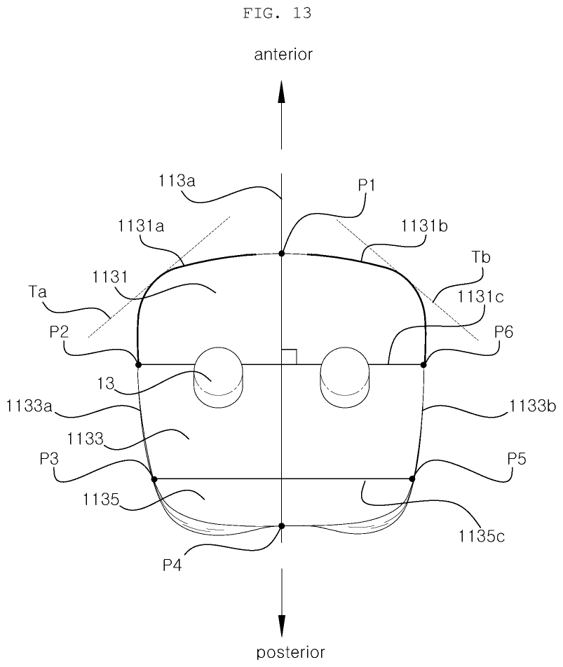

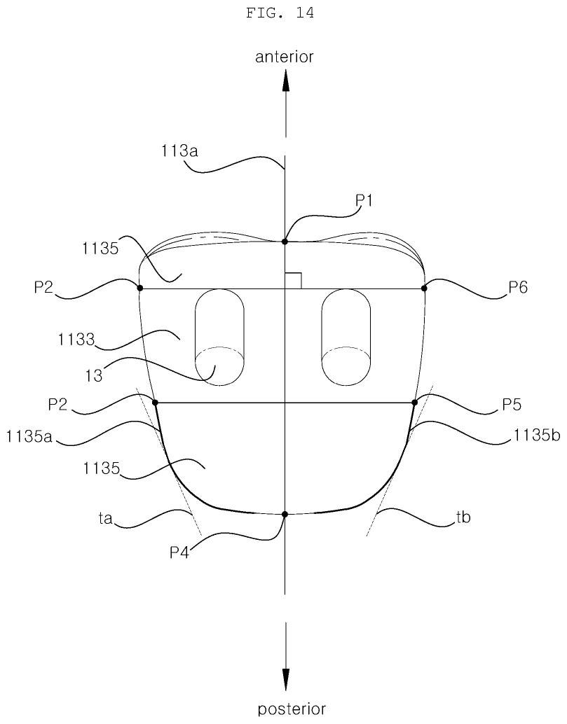

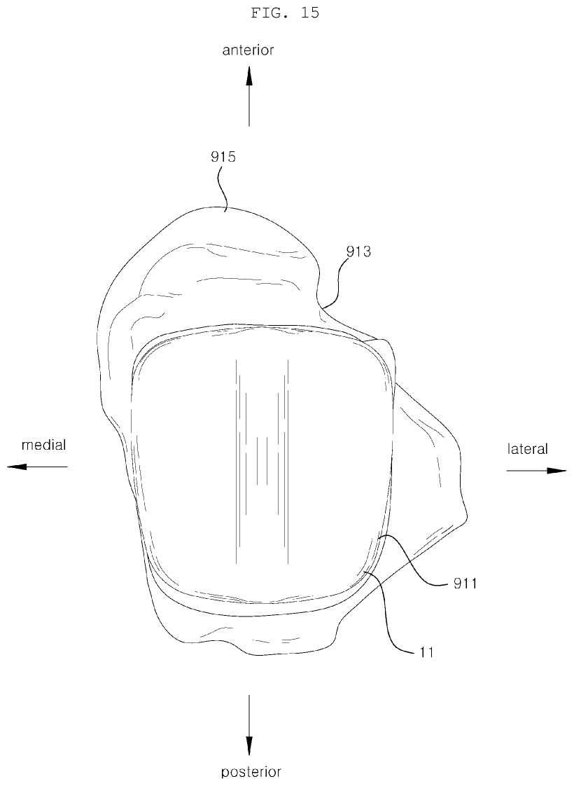

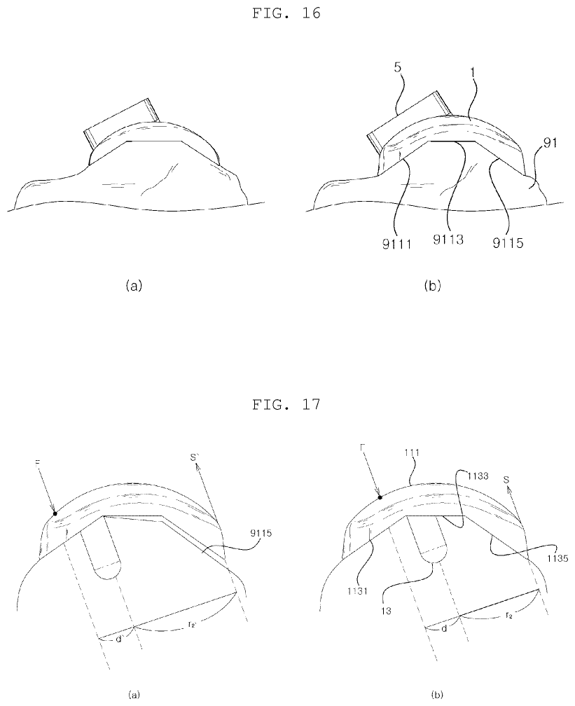

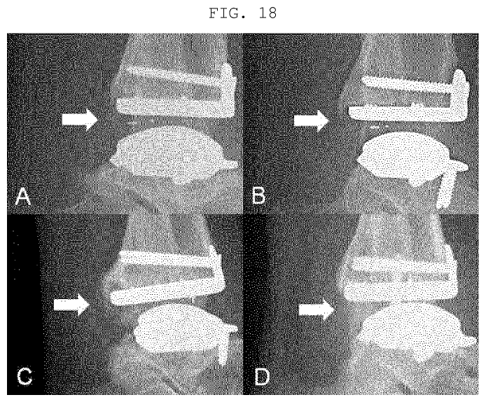

FIG. 1 is a plan view showing the state in which a talus component is coupled to a talus according to the prior art; FIG. 2 is a side view showing the case where a posterior portion of an implant lifts up when impacting a talus component with respect to a talus using a peg according to the prior art; FIG. 3 is a plan view and a side view showing the position and anatomy of a talus; FIG. 4 is a side view showing the state in which a talus component is coupled to a tibial component and an insert according to an embodiment of the present disclosure; FIG. 5 is a perspective view showing a talus component according to an embodiment of the present disclosure; FIG. 6 is a perspective view showing a talus component according to an embodiment of the present disclosure; FIG. 7 is a perspective view showing a talus component according to an embodiment of the present disclosure; FIG. 8 is a plan view showing a talus component according to an embodiment of the present disclosure; FIG. 9 is a front view showing a talus component according to an embodiment of the present disclosure; FIG. 10 is a rear view showing a talus component according to an embodiment of the present disclosure; FIG. 11 is a side view showing a talus component according to an embodiment of the present disclosure; FIG. 12 is a bottom view showing an intermediate surface according to an embodiment of the present disclosure; FIG. 13 is a bottom view showing an anterior surface according to an embodiment of the present disclosure; FIG. 14 is a bottom view showing a posterior surface according to an embodiment of the present disclosure; FIG. 15 is a plan view showing the state in which a talus component is coupled to a talus according to an embodiment of the present disclosure; FIG. 16 is a side view showing the movable range of an insert while a talus component is coupled to a talus according to an embodiment of the present disclosure; FIG. 17 is a cross-sectional side view showing the comparison a coupling process of a talus component according to the prior art with a coupling process of a talus component according to an embodiment of the present disclosure; and FIG. 18 is a view of radiation photos showing heterotopic ossification that may occur in artificial ankle joint arthroplasty according to the prior art.

Next, the position and anatomy of a talus will be described with reference to FIG. 3.

“(a)” of FIG. 3 is a side view showing a portion in which a talus 91 is positioned in an ankle joint. The talus 91 is positioned between a scaphoid 95, a tibia 93, and a calcaneus (heel bone) 97, and is in contact with a proximal end 931 below the tibia 93 to form an ankle joint, “(b)” of FIG. 3 is a side view showing only the talus 91 that is enlarged. The talus 91 is primarily including a talus body 911 in contact with the tibia 93, a talus head 915 in contact with the scaphoid 95, and a talus neck 913 connecting the body 911 and the head 915.

The talus body 911 is positioned in the upper portion of the talus 91, and has a talus fornix in the form of a side surface of a truncated cone. The talus fornix is in contact with the proximal end 931 of a tibia 93, and the tibia 93 moves forwards and backwards on the talus fornix, thereby performing dorsiflexion and plantar flexion motions. In general, in the case of artificial ankle joint arthroplasty, the upper portion of the talus fornix is resected in a certain range to form a cross-section corresponding to a talus component, and the talus component is inserted through the cross-section. Although the number of resected surfaces differs depending on products, a 3-surface tibia component for minimizing the amount of bone to be removed requires three resected surfaces, that is, an anterior resected surface, an intermediate resected surface, and a posterior resected surface. The anterior resected surface 9111 and the posterior resected surface 9115 are formed to be inclined forwards and backwards, respectively, along the bold line in “(b)” of FIG.3.

“(c)” of FIG. 3 is a plan view of the talus 91. The talus fornix has an anterior portion that is wider than a posterior portion thereof. However, an existing artificial ankle joint has the same width in the anterior portion and the posterior portion of the talus component, so that the talus component fails to accurately simulate the talus. In addition, although there is a slight difference in the structure thereof between people, the central portion of the talus fornix is formed to be slightly recessed, compared to the medial and lateral portions thereof, in order to provide an effective joint motion of the tibia 93.

Referring to the plan view of the talus fornix, the posterior boundary thereof is formed in a curve to be convex toward the posterior. Therefore, as will be described below, the posterior boundary 9115a of the posterior resected surface 9115, among the resected surfaces of the talus, is also formed to be curved toward the posterior. However, the talus component of an existing artificial ankle joint has a posterior boundary in the form of a recessed curve or a straight line, thereby failing to simulate the shape of the talus correctly.

In addition, the talus fornix has curved corners rather than right-angled corners. However, the talus component of the conventional artificial ankle joint is formed in a rectangle that does not conform to the shape of the resected surface of the talus.

Next, an artificial ankle joint system coupled to the talus component 1 to implement a joint motion of the ankle and a principle thereof will be briefly described with reference to FIG. 4.

An insert 5 made of plastic or the like and serving as a bearing is positioned on the talus component 1, and the talus component 1 slides back and forth along the curvature of the lower surface of the insert 5 in response to motion of the ankle, thereby implementing joint motions corresponding to dorsiflexion and plantar flexion motions. A tibial component 3, which is coupled to a distal end 933 of the tibia 93 and supports the load of the tibia 93, is positioned on the insert 5. The tibial component 3 may be a fixed type in which the tibial component 3 is immovably fixed to the insert 5, may be a semi-fixed type in which the tibial component 3 and the insert 5 partially restrict each other to allow a limited relative motion therebetween, or may be a free type in which the tibial component 3 is capable of unrestricted movement.

A combination of two or three components described above performs a joint motion in place of the ankle.

Now, the present disclosure will be described with reference to FIGS. 5 to 17.

Referring to FIGS. 5 and 6 illustrating the present disclosure, the talus component 1 of the present disclosure may include a body 11 that guides a bearing motion and is in contact with the talus 91 and pegs 13 extending from the body 11 to one side so as to be coupled to the talus 91.

Referring to FIG. 7, the body 11 may include a joint surface 111 in contact with the insert to implement a joint motion and a contact surface 113 coupled to the talus.

Referring to FIGS. 8 and 6, the joint surface 111 may be curved to have a curvature in the forwards and backwards direction in order to guide a joint motion of the insert 5. The curvature facilitates dorsiflexion that is the action of raising the foot upwards and plantar flexion that is the action of moving the foot points down after artificial ankle joint arthroplasty. In order to provide effective motion, the curvature may be preferably configured to be similar to the actual curvature of the talus fornix.

The joint surface 111 may include a medial joint surface 1111 close to the central side of a human body and a lateral joint surface 1115 close to a lateral side thereof. A connection joint surface 1113 positioned between the medial joint surface 1111 and the lateral joint surface 1115 may be formed to be recessed lower than the two surfaces 1111 and 1115. This can be confirmed in more detail in FIGS. 9 and 10. Such a configuration enables the insert 5, which will be described later, to stably move back and forth on the joint surface 111 without deviating in the medial/lateral direction during the joint motion. Connection portions between the medial and lateral joint surfaces 1111 and 1115 and the connection joint surface 1113 may be formed to have angled surfaces, or may be formed to have gentle curved surfaces.

The portions of the medial and lateral joint surfaces 1111 and 1115 at the edges in the medial and lateral directions may be formed to have angled surfaces, respectively, or may be more preferably formed to have gentle curved surfaces. This is due to the fact that the above-described prior art provides an excellent effect of preventing luxation of the insert 5 by forming the medial and lateral boundaries of the joint surface 111 to have a predetermined height, but has a disadvantage in that once luxation occurs, self-recovery is impossible, so that a surgical method for recovery is required. However, in the case where the medial and lateral boundaries are formed to be curved, if subluxation of the insert 5 occurs, natural recovery is possible.

In addition, the joint surface 111 may be preferably formed to extend to the edges of the contact surface 113 at the above curvature in the anterior and posterior/medial and lateral directions. The edges of the contact surface 113 may be formed along the resected surfaces of the talus fornix. If the joint surface 111 also extends the above edges, it is possible to maximize the movable range of the insert 5 and to provide effective distribution of stress.

In addition, the joint surface 111 meets the contact surface 113 at the boundaries thereof, and boundaries 1131a, 1131b, 1133a, 1133b, 1135a, and 1135b of the contact surface 113, which will be described later, are shared by the joint surface 111. Thus, the boundaries of the contact surface 113 may be regarded as the boundaries of the joint surface 111, so that the boundaries will be used to indicate the boundaries of both in the specification including the claims. However, it is clearer to describe the boundaries on the basis of the contact surface 113, and thus the following description will be made based on the same.

The detailed shape of the contact surface 113 will be described with reference to FIGS. 12 to 14.

Referring to FIG. 12, the distance between side edges 1133a, which are boundaries with respect to the medial and lateral portions, may be configured to increase as it goes from the posterior to the anterior. That is, the talus component 1 may be configured to have a truncated cone shape overall, in which the anterior portion is wider than the posterior portion. This is due to the fact that the posterior portion of the talus 91 is wider than the anterior portion thereof, as described above. The talus component 1 may have the shape complementary to the resected surfaces 9111, 9113, and 9115 of the talus 91 through the above configuration, thereby enabling close contact therebetween. Therefore, it is possible to prevent restriction of a movable range by a bone growing from the exposed resected surface and to maximize the contact area, thereby evenly distributing stress and improving the lifespan of the artificial ankle joint. Further, since the implant is designed to be more closely conform to the anatomical shape, a physiological joint implant may be obtained, thereby providing a comfortable joint after surgery.

Referring to FIG. 13, the contact surface 113 may include an anterior surface 1131 inclined in the anterior direction, a posterior surface 1135 inclined in the posterior direction, and an intermediate surface 1133 positioned therebetween.

The anterior surface 1131 may include an anterior lateral boundary 1131a at the lateral side thereof and an anterior medial boundary 1131b at the medial side thereof. The two anterior boundaries 1131a and 1131b may have a gentle arc shape, which is formed to be convex toward the anterior, thereby forming the shape complementary to the resected surface of the talus fornix. This is due to the fact that the anterior portion 9111 of the resected surface of the talus converges at the anterior.

At this time, if it is assumed that an anterior surface boundary 1131c, which is a boundary between the intermediate surface 1133 and the anterior surface 1131, corresponds to a horizontal axis, that a line 113a perpendicular to the horizontal axis and included in the anterior surface 1131 corresponds to a vertical axis, that the positive direction of the vertical axis corresponds to the anterior direction, and that the positive direction of the horizontal axis corresponds to the medial direction, the tangents of the anterior medial boundary 1131a and the anterior lateral boundary 1131b may continuously extend to the connection joint surface 1113 while having slopes opposite to each other, thereby leading to each other. Further, the slope of the tangent of each boundary may approach zero as it approaches the connection joint surface. Thus, the structure may conform to the anatomical shape of the talus by configuring the anterior boundaries to be gently curved as described above.

Accordingly, the anterior portion of the talus component 1 including the contact surface 113 and the joint surface 111 extending to the boundary thereof has a shape complementary to the anterior portion 9111 of the resected surface of the talus 91. In this structure, the talus implant may maximally cover the resected surfaces of the bone, thereby preventing osteolysis. Further, the above structure may prevent heterotopic ossification, and may maximize the contact area to evenly distribute stress, thereby preventing load concentration and prolonging the lifespan of the artificial joint.

Referring to FIG. 14, the posterior surface 1135 may include a posterior lateral boundary 1135a at the lateral side thereof and a posterior medial boundary 1135b at the medial side thereof. The two posterior boundaries 1135a and 1135b may have a gentle arc shape, which is formed to be convex toward the posterior, thereby forming the shape complementary to the resected surface of the talus fornix. This is due to the fact that the posterior portion 9115 of the resected surface of the talus converges at the posterior.

At this time, if it is assumed that a posterior surface boundary 1135c, which is a boundary between the intermediate surface 1133 and the posterior surface 1135, corresponds to a horizontal axis, that a line 113a perpendicular to the horizontal axis and included in the posterior surface 1135 corresponds to a vertical axis, that the positive direction of the vertical axis corresponds to the anterior direction, and that the positive direction of the horizontal axis corresponds to the medial direction, tangents of the posterior medial boundary 1135a and the posterior lateral boundary 1135b may continuously extend to the connection joint surface 1113 while having slopes opposite to each other, thereby leading to each other. Further, the slope of the tangent of each boundary may approximate zero as it approaches the connection joint surface. Thus, the structure may conform to the anatomical shape of the talus by configuring the posterior boundaries to be gently curved as described above.

Accordingly, the posterior portion of the talus component 1 including the contact surface 113 and the joint surface 111 extending to the boundary thereof has the shape complementary to the posterior portion 9115 of the resected surface of the talus 91. In addition, the above structure may prevent heterotopic ossification, and may maximize the contact area to evenly distribute stress, thereby preventing load concentration and prolonging the lifespan of the artificial joint.

Referring to FIGS. 11 and 17, pegs 13 may be formed to extend from the contact surface 113 to one side.

The peg 13 is an element that deeply penetrates into the bone by passing through the resected surface of the talus 91 to securely fix the talus component 1 to the talus 91. The pegs 13 are provided in each medial/lateral side, thereby preventing rotation thereof in the process of insertion.

A lower end 133 of the peg 13 may be configured in the form of a hemisphere, or may be configured to be a pointed figure such as a triangular pyramid, a quadrangular pyramid, or the like. In addition, any configuration may be applied to the lower end of the peg so as to enable insertion and fixation.

Preferably, the pegs 13 are formed to extend from the intermediate surface 1133 of the contact surface 113 to one side.

In artificial ankle joint arthroplasty, after a process of resecting a bone and selecting a size through trial, holes are formed in the talus 91, and the pegs 13 are inserted into the holes, thereby fixing the talus component 1. At this time, the peg 13 is inserted into the hole to a predetermined depth, and then a step of impacting the peg using a hammer or the like is performed to insert the same deeply, thereby fixing the talus component 1. At this time, if the peg 13 is positioned in the intermediate surface 1133, it is possible to prevent the posterior portion of the talus component 1 from being lifted up from the talus 91 by the rotation of the talus component 1 about the peg 13 in the anterior and posterior direction when impacting the same for insertion.

Now, a configuration for preventing the posterior portion from being lifted as described above will be described with reference to FIG. 17.

“A” shows a talus component, and “B” shows the present disclosure in which the peg 13 is positioned in the intermediate surface 1133.

Referring to FIG. 17, since an actual implant is small and ankle joint arthroplasty is performed through a narrow resection site, it is not easy to accurately impact the correct position. Therefore, it frequently happens that a force is applied to the wrong position, as shown in the case of “A” and “B”. When a force is applied to a wrong position as described above, momentum based on the peg occurs. The momentum is proportional to the distance “d” to the peg and the force “F”, so that the talus component 1 rotates about the peg. Although the momentum occurs, if the distance (r2) from the peg to the posterior end is short, it is not a big problem. However, as in the prior art (A), if the peg is located in the anterior portion, and thus, if r2′ is large, the posterior portion of the talus component 1 lifts up to be separated from the posterior portion 9115 of the resected surface of the talus. In general, bone cement, which is an adhesive, is applied to the contact surface 113 of the talus component 1 to facilitate fixation prior to impacting, but, if the posterior portion thereof lifts up as described above, even if the adhesive is applied thereto, it does not contribute to bonding. In particular, during an operation procedure through a narrow incision, since the posterior portion of the talus component and the posterior portion of the resected surface of the talus are not visible at all, checking is required using an image intensifier. This may increase the risk of radiation exposure and infection of surgeons. Therefore, there is a need for a structure that allows the talus component 1 to be placed initially at the correct position of the talus.

As in the present disclosure (B), if the peg 13 is located in the intermediate surface 1133, the distance (r2) from the peg 13, which is the center of momentum, to the end thereof is short. Therefore, even if momentum occurs, the posterior portion is not lifted up much, thereby ensuring a strong connection with the talus 91. Even if the momenta in both cases are the same because the distance “d” from the force acting point to the peg is the same as “d′”, the degree of lifting of the posterior portion is different therebetween (S′>S).

The peg 13 may be formed to extend at an incline from the intermediate surface 1133 of the contact surface 113 in the posterior direction. In artificial ankle joint arthroplasty, an anterior approach method is primarily performed by resecting the anterior portion of the ankle. In this case, since the ankle is thinner than other parts, the anterior incision is very narrow. Thus, it is necessary to push and fix the talus component 1 through a narrow space from the front. Accordingly, if the peg 13 is formed to be inclined from the anterior to the posterior, easier insertion and impacting are possible. At this time, the inclination angle of the peg 13 is preferably about 60 to 70 degrees with respect to the intermediate surface 1133.

According to the above-described configuration, the talus component 1 has the shape complementary to the resected surface obtained by cutting the body 911 of the talus 91, thereby securing a firm connection without exposure. In addition, it is possible to prevent heterotopic ossification, and thus osteoysis may be prevented. In addition, it is possible to prolong the lifespan of the implant by increasing the contact area to thus evenly distribute stress.

Referring to FIG. 18, showing stepwise photographs of heterotopic ossification after artificial ankle joint arthroplasty, it can be seen that unnecessary bone is formed in the posterior portion in “C” and “D”. This bone growth may limit the range of motion of the ankle joint, cause pain, and reduce the lifespan of the artificial ankle joint due to osteolysis by means of wear particles resulting from abrasion.

Although the above description has been made on the basis only of the talus component used in the artificial ankle joint, the description may also be applied to a tibia component, and the present disclosure may also be applied to implants used in artificial knee joints, artificial hip joints, artificial shoulder joints, or the like.

The above detailed description illustrates an example of the present disclosure. In addition, the above description relates to a preferred embodiment of the present disclosure, and the present disclosure may be used in various other combinations, modifications, and environments. That is, the present disclosure may be changed or modified within the scope of the concept of the disclosure disclosed in the present specification, the scope equivalent to the disclosed content, and/or the scope or knowledge of the art. The above-described embodiment illustrates the best mode for carrying out the technical idea of the present disclosure, and various modifications required for a specific application field and usage of the present disclosure are possible. Therefore, the detailed description of the disclosure above is not intended to limit the present disclosure to the disclosed embodiment. Further, the appended claims must be construed to encompass other embodiments.

1. An implant that is implanted into a body, the implant comprising a contact surface having a shape complementary to a resected surface of a bone into which the implant is implanted so as to increase contact area with respect to the bone, thereby distributing stress and mitigating side effects after surgery.

2. The implant of claim 1, wherein the implant is a talus component coupled to a talus in artificial ankle joint arthroplasty,
wherein the talus component comprises a joint surface in contact with an insert, and
wherein the joint surface is formed to have a curvature in an anterior and posterior direction so as to enable a joint motion of an ankle.

3. The implant of claim 2, wherein the joint surface comprises a medial joint surface positioned at a medial side, a lateral joint surface positioned at a lateral side, and a connection joint surface for connecting the medial joint surface and the lateral joint surface.

4. The implant of claim 3, wherein a posterior boundary of the contact surface has a gentle arc shape, which is formed to be convex toward the posterior, and
wherein the joint surface extends to the posterior boundary of the contact surface toward the posterior while having a curvature to maintain a large contact area with the talus, thereby distributing stress, mitigating side effects after surgery, and enabling a motion in a wide range.

5. The implant of claim 3, wherein an anterior boundary of the contact surface has a gentle arc shape, which is formed to be convex toward the anterior, and
wherein the joint surface extends to the anterior boundary of the contact surface toward the anterior while having a curvature to maintain a large contact area with the talus, thereby distributing stress, mitigating side effects after surgery, and enabling a motion in a wide range.

6. The implant of claim 3, wherein a posterior boundary of the contact surface has a gentle arc shape, which is formed to be convex toward the posterior,
wherein the joint surface extends to the posterior boundary of the contact surface toward the posterior while having a curvature,
wherein an anterior boundary of the contact surface has a gentle arc shape, which is formed to be convex toward the anterior, and
wherein the joint surface extends to the anterior boundary of the contact surface toward the anterior while having a curvature to maintain a large contact area with the talus, thereby distributing stress, mitigating side effects after surgery, and enabling a motion in a wide range.

7. The implant of claim 4, wherein tangents of a posterior medial boundary of the medial joint surface and a posterior lateral boundary of the lateral joint surface continuously extend toward the connection joint surface while having slopes opposite to each other and lead to each other.

8. The implant of claim 5, wherein tangents of an anterior medial boundary of the medial joint surface and an anterior lateral boundary of the lateral joint surface continuously extend toward the connection joint surface while having slopes opposite to each other and lead to each other.

9. The implant of claim 6, wherein tangents of a posterior medial boundary of the medial joint surface and a posterior lateral boundary of the lateral joint surface continuously extend toward the connection joint surface while having slopes opposite to each other and lead to each other, and
wherein tangents of an anterior medial boundary of the medial joint surface and an anterior lateral boundary of the lateral joint surface continuously extend toward the connection joint surface while having slopes opposite to each other and lead to each other.

10. The implant of claim 7, wherein the slope of the tangent of each boundary approaches zero as it approaches the connection joint surface.

11. The implant of claim 2, wherein the talus component is formed in a truncated cone shape in which the width thereof increases as it goes from the posterior to the anterior thereof so as to have a shape complementary to the resected surface of the talus, thereby minimizing an amount of bone to be removed, increasing the contact area to distribute stress, and mitigating side effects after surgery.

12. The implant of claim 2, wherein the contact surface comprises an anterior surface inclined to one side in the anterior portion, an intermediate surface formed in a plane at the center thereof, and a posterior surface inclined to one side in the posterior portion, thereby minimizing an amount of bone to be removed.

13. The implant of claim 12, further comprising a peg extending from the intermediate surface to a distal end.

14. The implant of claim 13, wherein the peg extends to be inclined at a predetermined angle toward the posterior.

15. The implant of claim 14, wherein the peg is inclined at 60 to 70 degrees toward the posterior.
